(19) United States
(12) Patent Application Publication
(10) Pub. No.: US 2024/0164736 A1
(43) Pub. Date: May 23, 2024

(54) METHODS, SYSTEMS, STORAGE MEDIA AND APPARATUS FOR TRAINING A BIOMEDICAL IMAGE ANALYSIS MODEL TO INCREASE PREDICTION ACCURACY OF A MEDICAL OR COSMETIC CONDITION

(71) Applicant: ONCORADIOMICS, Liege (BE)

(72) Inventor: Zerka FADILA, Liege (BE)

(73) Assignee: ONCORADIOMICS, Liege (BE)

(21) Appl. No.: 18/284,148

(22) PCT Filed: Jul. 30, 2022
(86) PCT No.: PCT/EP2022/071480

§ 371 Date: Sep. 26, 2023

(30) Foreign Application Priority Data
Aug. 4, 2021 (BE) 2021/5624

(51) Int. Cl. A61B 6/00; G06T 7/00; G16H 50/20; G16H 50/50

(52) U.S. Cl. CPC A61B 6/5217; A61B 6/563; G06T 7/0012; G16H 50/20; G16H 50/50; G06T 2207/10081; G06T 2207/10088; G06T 2207/10104; G06T 2207/20081; G06T 2207/30061

(57) ABSTRACT

Methods, systems, storage media and apparatus for training a biomedical image analysis model to increase prediction accuracy of a medical or cosmetic condition are disclosed. Some embodiments may include: providing an initial computer-implemented biomedical image analysis model having an initial model loss, providing one or more nodes configured to retrieve biomedical image data, and configured to execute the computer-implemented biomedical image analysis model, providing a smart contract infrastructure allowing for secured model weights exchange between the nodes, receiving a request by a node or sending a request to a node to train the computer-implemented biomedical image analysis model, training the computer-implemented biomedical image analysis model on the biomedical image data to obtain a modified biomedical image analysis model, calculating the model loss of the modified biomedical image analysis model and assessing the model loss of the modified biomedical image analysis model as compared to the model loss of the initial biomedical image analysis model.

Biomedical imaging is a non-invasive and an efficient tool for diagnosis, prog-nosis, treatment planning, and therapy and particularly useful in precision medicine.

The predictive value of the imaging models depends upon the quality and mostly quantity of the data used to train those models. Ideally, the data is grouped in a single database and processed immediately. However, ethical and legal consider-ations have considerably slowed down the initiatives of publicly sharing the biomedical imaging data.

Collecting imaging data into one database is technically challenging, costly, and requires considerable human resources to maintain and monitor the database. Moreover, a single institution cannot hold enough data to train generalizable predictive models. Moreover, traceability of data usage is a concern. Therefore, there is a need to provide an improved method of training biomedical image analysis models that addresses these concerns.

### FIELD OF THE DISCLOSURE

Accordingly, the present disclosure relates to methods, systems, storage media and apparatus for training of a biomedical image analysis model to increase prediction accuracy of a medical or cosmetic condition.

### BACKGROUND

The use of blockchain in medical technology is proposed for example in U.S. Ser. No. 11/043,307B2 granted to SMURRO JAMES PAUL. However, radiomics and blockchain are only disclosed amongst many other claim features and embodiments as part of a method and system for cognitive collaboration with neurosynaptic imaging networks, augmented medical intelligence and cybernetic workflow streams. The use of a smart contract infrastructure for distributed training is not disclosed.

US2020111578A1 to Radect discloses a reinforcement learning framework. Medical imaging with deep radiomics are described in general amongst a vast amount of embodiments. The use of a smart contract infrastructure for distributed training is not disclosed.

EP3786872A1 to Accenture discloses smart contract management in general. However, the use of a smart contract infrastructure for training of biomedical imaging analysis models is not disclosed.

### SUMMARY

The inventor now has surprisingly found, that the ERC 271 may be used to secure access to local biomedical image data and thus train biomedical image data analysis models on local biomedical image data whilst preserving both data security and availability. A decentralized privacy preserving distributed learning infrastructure is built upon blockchain technology to increase trust amongst network partners. This ensures both data security and traceability. The distributed learning network involves multiple partners. Each participating partner registers to the network's smart contract to participate in the model training using their local biomedical image data.

Accordingly, one aspect of the present disclosure relates to a method for training a biomedical image analysis model to increase prediction accuracy of a medical or cosmetic condition. The method may include providing an initial computer-implemented biomedical image analysis model to a node to generate an initial model loss. The method may include providing one or more nodes configured to retrieve model weights trained on biomedical image data stored in a cloud storage to a further node, configured to execute the computer-implemented biomedical image analysis model. The method may include providing a smart contract infrastructure allowing for secured model weights exchange between the first node and the further nodes. The method may include receiving a request by a node or sending a request to a node to train the computer-implemented biomedical image analysis model. The method may include training the computer-implemented biomedical image analysis model on the biomedical image data to obtain a modified biomedical image analysis model. The method may include calculating the model loss of the modified biomedical image analysis model. The method may include assessing the model loss of the modified biomedical image analysis model as compared to the model loss of the initial biomedical image analysis model.

In another aspect, the method further comprises the step of predicting a medical or cosmetic condition through the improved biomedical image model, wherein the prediction accuracy is improved as compared to the current model.

In another aspect, the medical condition is cancer, in particular lung cancer.

In another aspect, the biomedical image data is obtained by computed tomography, magnetic resonance imaging, or positron emission tomography.

In another aspect, the prediction accuracy is calculated as the Area Under the Curve of the Receiver Operating Characteristic curve.

In another aspect, the model loss is defined as a number indicating the loss of the model in terms of prediction accuracy as compared to the observed reality or ground truth.

In another aspect, the smart contract infrastructure is the Ethereum.

In another aspect, the biomedical image data model exchange between the first node and the furthernode is secured trough Ethereum Request for Comment 271.

Another aspect of the present disclosure relates to a system for training a biomedical image analysis model to increase prediction accuracy of a medical or cosmetic condition. The system may include one or more hardware processors configured by machine-readable instructions for training a biomedical image analysis model to increase prediction accuracy of a medical or cosmetic condition. The machine-readable instructions may be configured to provide an initial computer-implemented biomedical image analysis model having an initial model loss in a first node. The machine-readable instructions may be configured to provide one or more nodes configured to retrieve model weights trained on biomedical image data stored in a further node, and configured to execute the computer-implemented biomedical image analysis model. The machine-readable instructions may be configured to provide a smart contract infrastructure allowing for secured model weights exchange between the first node and the further nodes. The machine-readable instructions may be configured to receive a request by a node or sending a training request to a node to train the com-puter-implemented biomedical image analysis model. The machine-readable instructions may be configured to train the computer-implemented biomedical image analysis model on the biomedical image data to obtain a modified biomedical image analysis model. The machine-readable instructions may be configured to calculate the model loss of the modified biomedical image analysis model. The machine-readable instructions may be configured to assess the model loss of the modified biomedical image analysis model as compared to the model loss of the initial biomedical image analysis model.

Another aspect of the invention is a node, having access to biomedical image data comprising:
one or more hardware processors configured by machine-readable instructions to:
   execute a smart contract infrastructure allowing for secured model weights exchange between the first node and the node;
   receive a request by a node or sending a training request to a node to train the computer-implemented biomedical image analysis model;
   train the computer-implemented biomedical image analysis model on the biomedical image data to obtain a modified biomedical image analysis model;
   calculate the model loss of the modified biomedical image analysis model;
   assess the model loss of the modified biomedical image analysis model as compared to the model loss of the initial biomedical image analysis model, wherein if the model loss of the modified biomedical image analysis model be smaller than the initial biomedical image analysis model, sending of the modified biomedical image analysis model to the node or to the cloud to obtain an improved image analysis model, and wherein if the model loss of the modified biomedical image analysis model is smaller than the initial biomedical image analysis model, optionally repeating the preceding steps until the model loss of the modified biomedical image analysis model is smaller than the initial biomedical image analysis model.

Another aspect of the invention is node comprising a current biomedical image analysis model and a biomedical image computing unit configured to perform the method of the invention.

Another aspect of the present disclosure relates to a computer-readable storage medium for storing a biomedical image analysis model and model weights. In some embodiments, the computer-readable storage medium may include instructions being executable by one or more processors to provide an initial computer-implemented biomedical image analysis model having an initial model loss in a first node. In some embodiments, the computer-readable storage medium may include instructions being executable by one or more processors to provide one or more nodes configured to retrieve model weights trained on biomedical image data stored in a further node, and configured to execute the computer-implemented biomedical image analysis model. In some embodiments, the computer-readable storage medium may include instructions being executable by one or more processors to provide a smart contract infrastructure allowing for secured storage of bibliographic data that may consist of: node name, model name (for the first node), model weights name, model loss, or the models accuracy on the local test data, model accuracy on global test data (if available), iteration number. In some embodiments, the computer-readable storage medium may include instructions being executable by one or more processors to receive a request by a node or sending a training request to a node to train the computer-implemented biomedical image analysis model. In some embodiments, the computer-readable storage medium may include instructions being executable by one or more processors to train the computer-implemented biomedical image analysis model on the biomedical image data to obtain a modified biomedical image analysis model. In some embodiments, the computer-readable storage medium may include instructions being executable by one or more processors to calculate the model loss of the modified biomedical image analysis model. In some embodiments, the computer-readable storage medium may include instructions being executable by one or more processors to assess the model loss of the modified biomedical image analysis model as compared to the model loss of the initial biomedical image analysis model.

Another aspect of the present disclosure relates to an apparatus configured for training a biomedical image analysis model to increase prediction accuracy of a medical or cosmetic condition. In some aspects, the apparatus may include at least one memory storing computer program instructions and at least one processor configured to execute the computer program instructions to cause the apparatus at least to perform operations associated with training a biomedical image analysis model to increase prediction accuracy of a medical or cosmetic condition. In some aspects, the computer program instructions may include providing an initial computer-implemented biomedical image analysis model having an initial model loss in a first node. In some aspects, the computer program instructions may include providing one or more nodes configured to retrieve model weights trained on biomedical image data stored in a further cloud storage to a node configured to execute the computer-implemented biomedical image analysis model. In some aspects, the computer program instructions may include providing a smart contract infrastructure allowing for secured model weights exchange between the first node and the further nodes. In some aspects, the computer program instructions may include receiving a request by a node or sending a training request to a node to train the computer-implemented biomedical image analysis model. In some aspects, the computer program instructions may include training the computer-implemented biomedical image analysis model on the biomedical image data to obtain a modified biomedical image analysis model. In some aspects, the computer program instructions may include calculating the model loss of the modified biomedical image analysis model. In some aspects, the computer program instructions may include assessing the model loss of the modified biomedical image analysis model as compared to the model loss of the initial biomedical image analysis model.

The methods and systems of the present inventions allow for improved prediction accuracy of biomedical image analysis models through training on decentral biomedical image data whilst guaranteeing confidentiality of sensitive medical data through smart contracts.

### BRIEF DESCRIPTION OF THE DRAWINGS

FIG. 1 illustrates a system configured for training a biomedical image analysis model to increase prediction accuracy of a medical or cosmetic condition.

FIG. 2 illustrates a method for training a biomedical image analysis model to increase prediction accuracy of a medical or cosmetic condition.

FIG. 3 illustrates a system for training a biomedical image analysis model to increase prediction accuracy of a medical or cosmetic condition:

### DETAILED DESCRIPTION

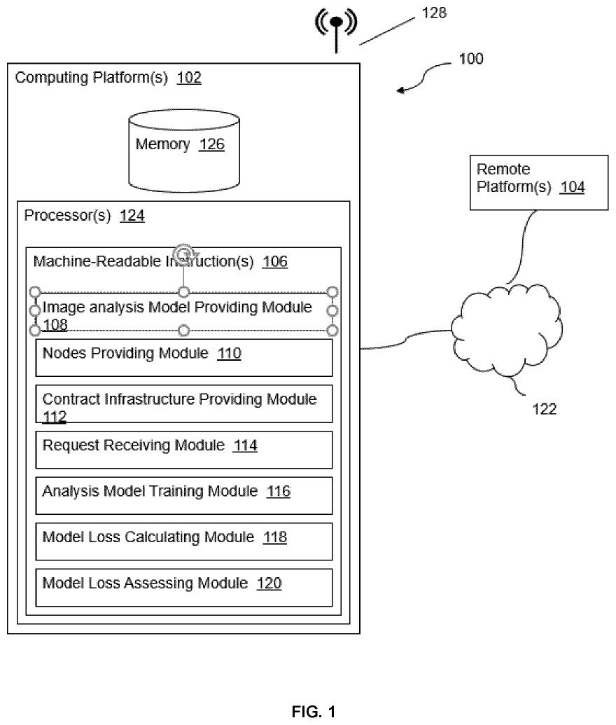

FIG. 1 illustrates a system configured for training a biomedical image analysis model to increase prediction accuracy of a medical or cosmetic condition, in accord-ance with one or more embodiments. In some cases, system 100 may include one or more computing platforms 102. The one or more remote computing platforms 102 may be communicably coupled with one or more remote platforms 104. In some cases, users may access the system 100 via remote platform(s) 104.

The one or more computing platforms 102 may be configured by machine-readable instructions 106. Machine-readable instructions 106 may include modules. The modules may be implemented as one or more of functional logic, hardware logic, electronic circuitry, software modules, and the like. The modules may include one or more of analysis model providing module 108, nodes providing module 110, contract infrastructure providing module 112, request receiving module 114, analysis model training module 116, model loss calculating module 118, model loss assessing module 120, and/or other modules.

Analysis model providing module 108 may be configured to provide an initial computer-implemented biomedical image analysis model having an initial model loss in a first node. Nodes providing module 110 may be configured to provide one or more nodes configured to retrieve model weights trained on biomedical image data stored in a further, and configured to execute the computer-implemented biomedical image analysis model. Contract infrastructure providing module 112 may be configured to provide a smart contract infrastructure allowing for secured model weights exchange between the first node and the further nodes. Request receiving module 114 may be configured to receive a request by a node or sending a training request to a node to train the computer-implemented biomedical image analysis model. Analysis model training module 116 may be configured to train the computer-implemented biomedical image analysis model on the biomedical image data to obtain a modified biomedical image analysis model. Model loss calculating module 118 may be configured to calculate the model loss of the modified biomedical image analysis model. Model loss assessing module 120 may be configured to assess the model loss of the modified biomedical image analysis model as compared to the model loss of the initial biomedical image analysis model. If the model loss of the modified biomedical image analysis model is smaller than the initial biomedical image analysis model, sending of the modified biomedical image analysis model to the following node to obtain an improved image analysis model, and If the model loss of the modified biomedical image analysis model may be smaller than the initial biomedical image analysis model, optionally repeating the preceding steps until the model loss of the modified biomedical image analysis model may be smaller than the initial biomedical image analysis model.

In some cases, the one or more computing platforms 102, may be communi-catively coupled to the remote platform(s) 104. In some cases, the communicative coupling may include communicative coupling through a networked environment 122. The networked environment (Cloud storage) 122 may be a radio access network, such as LTE or 5G, a local area network (LAN), a wide area network (WAN) such as the Internet, or wireless LAN (WLAN), for example. It will be appreciated that this is not intended to be limiting, and that the scope of this disclosure includes implemen-tations in which one or more computing platforms 102 and remote platform(s) 104 may be operatively linked via some other communication coupling. The one or more one or more computing platforms 102 may be configured to communicate with the networked environment (Cloud storage) 122 via wireless or wired connections. In addition, in an embodiment, the one or more computing platforms 102 may be configured to communicate directly with each other via wireless or wired connections. Examples of one or more computing platforms 102 may include, but is not limited to, smartphones, wearable devices, tablets, laptop computers, desktop computers, Internet of Things (IOT) device, or other mobile or stationary devices. In an embodiment, system 100 may also include one or more hosts or servers, such as the one or more remote platforms 104 connected to the networked environment (Cloud storage) 122 through wireless or wired connections. According to one embodiment, remote platforms 104 may be implemented in or function as base stations (which may also be referred to as Node Bs or evolved Node Bs (eNBs)). In other embodiments, remote platforms 104 may include web servers, mail servers, application servers, etc. According to certain embodiments, remote platforms 104 may be standalone servers, networked servers, or an array of servers.

The one or more computing platforms 102 may include one or more processors 124 for processing information and executing instructions or operations. One or more processors 124 may be any type of general or specific purpose processor. In some cases, multiple processors 124 may be utilized according to other embodiments. In fact, the one or more processors 124 may include one or more of general-purpose computers, special purpose computers, microprocessors, digital signal processors (DSPs), field-programmable gate arrays (FPGAs), application-specific inte-grated circuits (ASICs), and processors based on a multi-core processor architecture, as examples. In some cases, the one or more processors 124 may be remote from the one or more computing platforms 102, such as disposed within a remote platform like the one or more remote platforms 124 of FIG. 1.

The one or more processors 124 may perform functions associated with the operation of system 100 which may include, for example, precoding of antenna gain/phase parameters, encoding and decoding of individual bits forming a communication message, formatting of information, and overall control of the one or more computing platforms 102, including processes related to management of communication resources.

The one or more computing platforms 102 may further include or be coupled to a memory 126 (internal or external), which may be coupled to one or more processors 124, for storing information and instructions that may be executed by one or more processors 124. Memory 126 may be one or more memories and of any type suitable to the local application environment, and may be implemented using any suitable volatile or nonvolatile data storage technology such as a semiconductor-based memory device, a magnetic memory device and system, an optical memory device and system, fixed memory, and removable memory. For example, memory 126 can consist of any combination of random access memory (RAM), read only memory (ROM), static storage such as a magnetic or optical disk, hard disk drive (HDD), or any other type of non-transitory machine or computer readable media. The instructions stored in memory 126 may include program instructions or computer program code that, when executed by one or more processors 124, enable the one or more computing platforms 102 to perform tasks as described herein.

In some embodiments, one or more computing platforms 102 may also include or be coupled to one or more antennas for transmitting and receiving signals and/or data to and from one or more computing platforms 102. The one or more antennas may be configured to communicate via, for example, a plurality of radio interfaces that may be coupled to the one or more antennas. The radio interfaces may corre-spond to a plurality of radio access technologies including one or more of LTE, 5G, WLAN, Bluetooth, near field communication (NFC), radio frequency identifier (RFID), ultrawideband (UWB), and the like. The radio interface may include components, such as filters, converters (for example, digital-to-analog converters and the like), mappers, a Fast Fourier Transform (FFT) module, and the like, to generate symbols for a transmission via one or more downlinks and to receive symbols (for example, via an uplink).

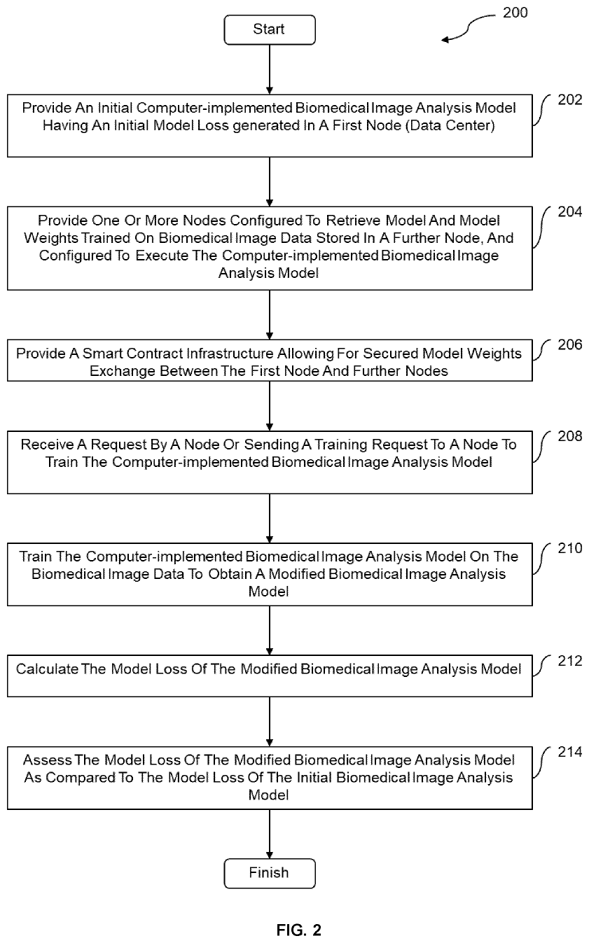

FIG. 2 illustrate an example flow diagram of a method 200, according to one embodiment. The method 200 may include providing an initial computer-implemented biomedical image analysis model having an initial model loss in a first node at block 202. The method 200 may include providing one or more nodes configured to retrieve biomedical image data stored in a further node, and configured to execute the com-puter-implemented biomedical image analysis model at block 204. The method 200 may include providing a smart contract infrastructure allowing for secured model weights exchange between the first node and the further nodes at block 206. The method 200 may include receiving a request by a node or sending a training request to a node to train the computer-implemented biomedical image analysis model at block 208. The method 200 may include training the computer-implemented biomedical image analysis model on the biomedical image data to obtain a modified biomedical image analysis model at block 210. The method 200 may include calculating the model loss of the modified biomedical image analysis model at block 212. The method 200 may include assessing the model loss of the modified biomedical image analysis model as compared to the model loss of the initial biomedical image analysis model at block 214, if the model loss of the modified biomedical image analysis model being smaller than the initial biomedical image analysis model, sending of the modified biomedical image analysis model to the further node to obtain an improved image analysis model, and if the model loss of the modified biomedical image analysis model may be smaller than the initial biomedical image analysis model, optionally repeating the preceding steps until the model loss of the modified biomedical image analysis model may be smaller than the initial biomedical image analysis model.

In some cases, the method 200 may be performed by one or more hardware processors, such as the processors 124 of FIG. 1, configured by machine-readable instructions, such as the machine readable instructions 106 of FIG. 1. In this aspect, the method 200 may be configured to be implemented by the modules, such as the modules 108, 110, 112, 114, 116, 118 and/or 120 discussed above in FIG. 1.

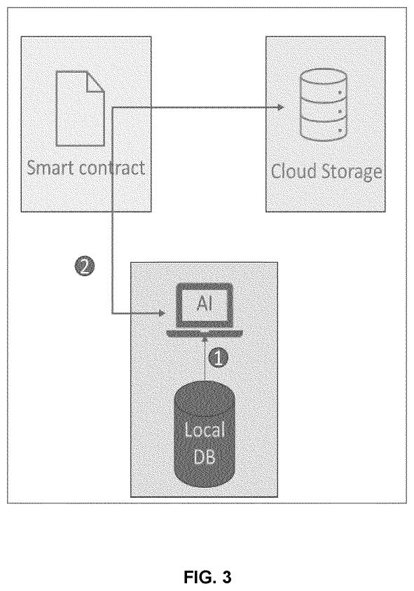

FIG. 3 illustrates a further embodiment of the method and system of the present invention comprising steps 1 and 2 that are executed in consecutive order configured to sequentially perform the following steps and in the following order:

In a first step, an ERC 271 Blockchain token is generated for the node.

After the node finishes the model training, the smart contract compares the loss of the current model (estimated for the local validation data) to the loss of the previous iteration (trained using data of previous node).

If the current loss is more than the previous loss, the node is moved to a new list “newChance” to wait for a second chance. This process follows the logic of trans-fer learning. After updating the model with new data, the model might be able to capture features in the discarded node data that it could not capture before.

The current model is saved to the cloud to optimize the mining process and transaction costs.

The current token is burnt.

The training iteration allows the next node to update the model.

A new token is generated for the next node to update the model using local data.

After 48 hours, if a model is not ready for assessment, for example for a hardware issue or internet shortage, then the following steps are performed:

The process starting from the first step is repeated, until all nodes get a chance to update the model with their data.

When the current node restarts the method, it is checked if it uploaded a model to cloud. If not, the corresponding token is burnt and the process is repeated.

After all the nodes got a chance to update the model with their local data, re-peat the process from step 1 for the nodes stored in the “newChance” list.

If the model is not validated, then the node is moved to a “freezer” list where it waits for new nodes to join the network, in order to get a chance to update and vali-date the model using their local data.

The methods and systems of the invention ensure secured access to locally stored biomedical image data whilst preserving data security and confidentiality of sensitive medical biomedical image information.

### EXAMPLES

Example 1 includes a method comprising: providing an initial computer-implemented biomedical image analysis model having an initial model loss in a first node, providing one or more nodes configured to retrieve model weights trained on biomedical image data stored in a further node, and configured to execute the computer-implemented biomedical image analysis model, providing a smart contract infrastructure allowing for secured model weights exchange between the first node and the further nodes, receiving a request by a node or sending a training request to a node to train the computer-implemented biomedical image analysis model, training the computer-implemented biomedical image analysis model on the biomedical image data to obtain a modified biomedical image analysis model, calculating the model loss of the modified biomedical image analysis model and assessing the model loss of the modified biomedical image analysis model as compared to the model loss of the initial biomedical image analysis model.

Example 2 includes a system comprising: providing an initial computer-implemented biomedical image analysis model having an initial model loss in a first node, providing one or more nodes configured to retrieve model weights trained on biomedical image data stored in a further node, and configured to execute the computer-implemented biomedical image analysis model, providing a smart contract infrastructure allowing for secured model weights exchange between the first node and the further nodes, receiving a request by a node or sending a training request to a node to train the computer-implemented biomedical image analysis model, training the computer-implemented biomedical image analysis model on the biomedical image data to obtain a modified biomedical image analysis model, calculating the model loss of the modified biomedical image analysis model and assessing the model loss of the modified biomedical image analysis model as compared to the model loss of the initial biomedical image analysis model.

Example 3 includes a storage medium comprising: providing an initial com-puter-implemented biomedical image analysis model having an initial model loss in a first node, providing one or more nodes configured to retrieve biomedical image data stored in a further node, and configured to execute the computer-implemented biomedical image analysis model, providing a smart contract infrastructure allowing for secured model weights exchange between the first node and the further nodes, receiving a request by a node or sending a training request to a node to train the com-puter-implemented biomedical image analysis model, training the computer-implemented biomedical image analysis model on the biomedical image data to obtain a modified biomedical image analysis model, calculating the model loss of the modified biomedical image analysis model and assessing the model loss of the modified biomedical image analysis model as compared to the model loss of the initial biomedical image analysis model.

Example 4 includes an apparatus comprising: providing an initial computer-implemented biomedical image analysis model having an initial model loss in a first node, providing one or more nodes configured to retrieve biomedical image data stored in a further node, and configured to execute the computer-implemented biomedical image analysis model, providing a smart contract infrastructure allowing for secured model weights exchange between the first node and the further nodes, receiving a request by a node or sending a training request to a node to train the com-puter-implemented biomedical image analysis model, training the computer-implemented biomedical image analysis model on the biomedical image data to obtain a modified biomedical image analysis model, calculating the model loss of the modified biomedical image analysis model and assessing the model loss of the modified biomedical image analysis model as compared to the model loss of the initial biomedical image analysis model.

Example 5 illustrates the embodiment shown in FIG. 3. The open-source NSCLC-Radiomics dataset available at https://wiki.cancerimagingarchive.net/dis-play/Public/NSCLC-Radiomics was used to simulate a distributed learning network of four centers to predict a 2-year survival after treatment. The data consists of pretreat-ment CT scans and clinical outcome data of 422 NSCLC patients. Manual segmen-tations performed by a radiation oncologist of the 3D volume of the GTV are made available.

The data was split in 60% training and 20% validation and 20% testing. The training data was used to train the model, the validation data was used during the training to access the learning process and fine tune the model parameters, and the test data is used to evaluate the models' performance. The training data was further split into four equal portions (Center 1, Center 2, Center 3, Center 4) used to train the distributed models. Each center data was placed in a folder accessible by the software. The order of training is determined by the order of launching the software, for example: if Center 1 starts the software first, then center 3, followed by Center 4, and Center 2, then training order would be: 1) Center 1; 2) Center 3; 3) Center 4; 4) Center 2. In this use case the training order was as follow: 1) Center 1; 2) Center 2; 3) Center 3; 4) Center 4.

The following results were obtained:

Model AUC

Center 1 0.7

Center 2 0.75

Center 3 0.77

Center 4 Not uploaded as it did not improve the model from Center 3

### Abbreviations

ERC: Ethereum Request for Comment

ERC 271: a non-fungible token standard where each token is unique and can have different values. This makes it useful for representing physical property and other such assets. ERC-721 tracks ownership of each token individually. Addition-ally, tokens can be deleted, and associated methods are robust against faulty in-puts. However, it does not provide any type of data structure to associate tokens with individual properties.

Model loss: the penalty for a bad prediction (model loss is a number indicating how bad the model's prediction was on a single example). If the model's prediction is perfect, the loss is zero; otherwise, the loss is greater.

AUC: Area Under the Curve (AUC) of the Receiver Operating Characteristic curve (ROC)

CT: Computed Tomography

GTV: Gross Tumor Volume

NSCLC: Non Small Cell Lung Cancer

Translation of the English Expressions in the Drawings

Smart contract contrat intelligent

Cloud storage stockage en nuage

Local DB base de données local

AI intelligence artificielle

1. A method comprising:
providing an initial computer-implemented biomedical image analysis model having an initial model loss;
providing one or more nodes configured to retrieve biomedical image data, and configured to execute the computer-implemented biomedical image analysis model;
providing a smart contract infrastructure allowing for secured model weights exchange between the nodes;
receiving a request by a node or sending a request to a node to train the computer-implemented biomedical image analysis model;
training the computer-implemented biomedical image analysis model on the biomedical image data to obtain a modified biomedical image analysis model;
calculating the model loss of the modified biomedical image analysis model;
assessing the model loss of the modified biomedical image analysis model as compared to the model loss of the initial biomedical image analysis model,
wherein, if the model loss of the modified biomedical image analysis model is smaller than the initial biomedical image analysis model, sending of the model weights of the modified biomedical image analysis model to the node or to the cloud to obtain an improved image analysis model,
and wherein, if the model loss of the modified biomedical image repeating the preceding steps until the model loss of the modified biomedical image analysis model is smaller than the initial biomedical image analysis model.

2. The method of claim 1, further comprising the step of predicting a medical or cosmetic condition through the improved biomedical image model, wherein the prediction accuracy is improved as compared to the current model.

3. The method of claim 1, wherein the medical condition is cancer, in particular lung cancer.

4. The method of claim 1, wherein the biomedical image data is obtained by computed tomography, magnet resonance imaging, or positron emission tomography.

5. The method of claim 1, wherein the prediction accuracy is calculated as the Area Under the Curve of the Receiver Operating Characteristic curve.

6. The method of claim 1, wherein the model loss is defined as a number indicating the loss of the model in terms of prediction accuracy as compared to the observed reality or ground truth.

7. The method of claim 1, wherein the smart contract infrastructure is the Ethereum.

8. The method of claim 1, wherein the biomedical image model weights exchange between the first node and the node is secured trough Ethereum Request for Comment 271.

9. A system comprising one or more hardware processors configured by machine-readable instructions to:
provide an initial computer-implemented biomedical image analysis model having an initial model loss;
provide one or more nodes configured to retrieve biomedical image data stored in a further node, and configured to execute the computer-implemented biomedical image analysis model;
provide a smart contract infrastructure allowing for secured model weights exchange between the first node and the further nodes;
receive a request by a node or sending a request to a node to train the computer-implemented biomedical image analysis model;
train the computer-implemented biomedical image analysis model on the biomedical image data to obtain a modified biomedical image analysis model;
calculate the model loss of the modified biomedical image analysis model;
assess the model loss of the modified biomedical image analysis model as compared to the model loss of the initial biomedical image analysis model,
wherein if the model loss of the modified biomedical image analysis model is smaller than the initial biomedical image analysis model, sending of the modified biomedical image analysis model to the node or to the cloud to obtain an improved image analysis model,
and wherein if the model loss of the modified biomedical image repeating the preceding steps until the model loss of the modified biomedical image analysis model is smaller than the initial biomedical image analysis model.

10. A node having access to biomedical image data comprising one or more hardware processors configured by machine-readable instructions to:
execute a smart contract infrastructure allowing for secured model weights exchange between the first node and the node;
receive a request by a node or sending a request to a node to train the computer-implemented biomedical image analysis model;
train the computer-implemented biomedical image analysis model on the biomedical image data to obtain a modified biomedical image analysis model;
calculate the model loss of the modified biomedical image analysis model;
assess the model loss of the modified biomedical image analysis model as compared to the model loss of the initial biomedical image analysis model,
wherein if the model loss of the modified biomedical image analysis model be smaller than the initial biomedical image analysis model, sending of the modified biomedical image analysis model to the node to obtain an improved image analysis model,
and wherein if the model loss of the modified biomedical image analysis model is smaller than the initial biomedical image analysis model, optionally repeating the preceding steps until the model loss of the modified biomedical image analysis model is smaller than the initial biomedical image analysis model.

11. A node comprising a current biomedical image analysis model and a biomedical image computing unit configured to perform the method of claim 1.

12. A non-transient computer-readable storage medium comprising instructions being executable by one or more processors to perform a method, the method comprising:
providing an initial computer-implemented biomedical image analysis model having an initial model loss in a first node;
providing one or more nodes configured to retrieve biomedical image data stored in a further node, and configured to execute the computer-implemented biomedical image analysis model;
providing a smart contract infrastructure allowing for secured model weights exchange between the first node and the further nodes;
receiving a request by a node or sending a training request to a node to train the computer-implemented biomedical image analysis model;
training the computer-implemented biomedical image analysis model on the biomedical image data to obtain a modified biomedical image analysis model;
calculating the model loss of the modified biomedical image analysis model;
assessing the model loss of the modified biomedical image analysis model as compared to the model loss of the initial biomedical image analysis model,
wherein if the model loss of the modified biomedical image analysis model being smaller than the initial biomedical image analysis model, sending of the modified biomedical image analysis model to the node or to the cloud to obtain an improved image analysis model,
and wherein if the model loss of the modified biomedical image repeating the preceding steps until the model loss of the modified biomedical image analysis model is smaller than the initial biomedical image analysis model.

13. An apparatus comprising:
at least one memory storing computer program instructions; and at least one processor configured to execute the computer program instructions to cause the apparatus at least to:
provide an initial computer-implemented biomedical image analysis model having an initial model loss in a first node;
provide one or more nodes configured to retrieve biomedical image data stored in a further node, and configured to execute the computer-implemented biomedical image analysis model;
provide a smart contract infrastructure allowing for secured model weights exchange between the first node and the further nodes;
receive a request by a node or sending a training request to a node to train the computer-implemented biomedical image analysis model;
train the computer-implemented biomedical image analysis model on the biomedical image data to obtain a modified biomedical image analysis model;
calculate the model loss of the modified biomedical image analysis model;
assess the model loss of the modified biomedical image analysis model as compared to the model loss of the initial biomedical image analysis model,
wherein if the model loss of the modified biomedical image analysis model be smaller than the initial biomedical image analysis model, sending of the modified biomedical image analysis model to the node or to the cloud to obtain an improved image analysis model,
and wherein if the model loss of the modified biomedical image analysis model is smaller than the initial biomedical image analysis model, optionally repeating the preceding steps until the model loss of the modified biomedical image analysis model is smaller than the initial biomedical image analysis model.
